(19) United States
(12) Patent Application Publication
(10) Pub. No.: US 2017/0333317 A1
(43) Pub. Date: Nov. 23, 2017

(54) DEODORIZING ACTIVE INGREDIENT COMBINATION

(71) Applicant: Henkel AG & Co. KGaA, Duesseldorf (DE)

(72) Inventor: THOMAS DOERING, DORMAGEN (DE)

(73) Assignee: Henkel AG & Co. KGaA, Duesseldorf (DE)

(21) Appl. No.: 15/593,357

(22) Filed: May 12, 2017

(30) Foreign Application Priority Data
May 23, 2016 (DE) 102016208856.4

(51) Int. Cl. A61K 8/41; A61K 8/37; A61Q 15/00

(52) U.S. Cl. CPC A61K 8/416; A61Q 15/00; A61K 8/37; A61K 2800/30; A61K 2800/74; A61K 2800/596

(57) ABSTRACT

An aqueous, deodorizing cosmetic agent which contains:
at least one quaternary ammonium compound and
triethyl citrate,
   wherein the deodorizing cosmetic agent contains no aluminum-containing compounds, use thereof, and method using such an agent.

### CROSS-REFERENCE TO RELATED APPLICATION

This application claims priority to German Application No. 10 2016 208 856.4, filed May 23, 2016, which are all hereby incorporated in their entirety by reference

### TECHNICAL FIELD

This application pertains to cosmetic deodorant compositions having an improved effect, and to methods using said compositions.

### BACKGROUND

In many cultural groups, the cleaning and care of one's own body includes measures intended to counteract the development of excessive body odor that is perceived as unpleasant. The development of body odor is brought about inter alia by perspiration, and therefore the cosmetic industry provides numerous body care products which have been developed for use in body areas having a high density of sweat glands, in particular in the underarm area. These body care products have a sweat-inhibiting effect (antiperspirant) and/or a deodorizing effect (deodorant).

Cosmetic antiperspirants of the prior art usually contain at least one antiperspirant salt in addition to at least one oil or one fatty substance and a fragrance component or a perfume. The antiperspirant salts used in said antiperspirants on the one hand reduce the secretion of sweat from the body by temporarily narrowing and/or blocking the efferent ducts of the sweat glands, so that the amount of sweat can be reduced by around 20 to 60 percent. On the other hand, due to their antimicrobial action, they have an additional deodorizing effect. Usually activated basic aluminum and aluminum-zirconium halides are used as antiperspirant salts. Aluminum and aluminum-zirconium halides which are stabilized with organic acids as complex ligands can also be used. People with sensitive skin can react to antiperspirant salts with skin irritations. Aluminum-free antiperspirants and deodorants are described for example in German patent applications DE102013225617 A1 and DE102013225620 A1.

Antiperspirants and deodorants can be prepared in various administration forms, for example as a powder, in stick form, as an aerosol spray, as a liquid or gel-like roll-on application, as a cream, as a gel and as impregnated flexible substrates (deodorant wipes).

Preparing the antiperspirants and deodorants in liquid form has advantages with regard to dosing and application, for example by means of an atomizer or spray. However, emulsion systems of low viscosity which are suitable for atomization often do not have a sufficient storage stability.

### BRIEF SUMMARY

Aqueous, deodorizing cosmetic agents for preventing and/or reducing body odor and methods for using such deodorizing cosmetic agents are provided herein. In an exemplary embodiment, an aqueous, deodorizing cosmetic agent includes at least one quaternary ammonium compound, and triethyl citrate. The deodorizing cosmetic agent contains no aluminum-containing compounds

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

Against the background, the technical problem addressed by the present application was that of providing a deodorant with high efficacy against body odor, good skin compatibility, and good storage stability and microbiological stability.

In an exemplary embodiment, it has now surprisingly been found that the aforementioned technical problems are solved by aluminum-free compositions which contain at least one quaternary ammonium compound in combination with triethyl citrate. The present disclosure provides an aqueous, deodorizing cosmetic agent which contains at least one quaternary ammonium compound and triethyl citrate. The deodorizing cosmetic agent contains no aluminum-containing compounds.

In one aspect, the agent contains as the quaternary ammonium compound at least one compound selected from the group consisting of behentrimonium chloride, cetrimonium bromide, cetrimonium chloride, laurtrimonium bromide, laurtrimonium chloride, steartrimonium bromide, steartrimonium chloride, benzethonium chloride, benzalkonium chloride, and mixtures thereof.

In another aspect, the agent contains benzalkonium chloride as the quaternary ammonium compound.

In yet another aspect, the quaternary ammonium compound is contained in an amount of from about 0.01 to about 0.3% by weight, preferably from about 0.02 to about 0.2% by weight and in particular from about 0.05 to about 0.1% by weight, based on the total weight of the agent.

In one aspect, the triethyl citrate is contained in an amount of from about 0.1 to about 5.0% by weight, preferably from about 0.25 to about 4.0% by weight and in particular from about 0.5 to about 3.0% by weight, based on the total weight of the agent.

In another aspect, the agent contains at least one bactericide selected from the group consisting of chlorhexidine, farnesol, triclosan, chloroxylenol and phenoxyethanol, preferably phenoxyethanol.

In yet another aspect, the agent contains about 0.1 to about 2.0% by weight, preferably about 0.2 to about 1.6% by weight and in particular about 0.5 to about 1.4% by weight of a bactericide, preferably phenoxyethanol.

In one aspect, the agent contains at least one nonionic emulsifier selected from the group consisting of the addition products of about 2 to about 50 mol ethylene oxide with linear fatty alcohols having 12 to 18 carbon atoms.

In another aspect, the agent contains the nonionic emulsifier in an amount of from about 1.0 to about 10% by weight, preferably from about 2.0 to about 8.0% by weight and in particular from about 3.0 to about 6.0% by weight, based on the total weight of the agent.

In yet another aspect, the agent contains at least one cellulose ether, preferably a cellulose ether from the group consisting of hydroxypropyl cellulose, hydroxyethyl cellulose and methyl hydroxypropyl cellulose, in particular hydroxyethyl cellulose.

In one aspect, the agent contains the cellulose ether, in particular the hydroxyethyl cellulose, in amounts of from about 0.1 to about 1.2% by weight, preferably from about 0.15 to about 1.0% by weight and in particular from about 0.2 to about 0.8% by weight, based on the total weight of the agent.

In another aspect, ethanol is contained in amounts between about 10 and about 50% by weight, preferably between about 20 and about 40% by weight and in particular between about 23 and about 35% by weight, based on the total weight of the agent.

In yet another aspect, water is contained in amounts of from about 45 to about 85% by weight, preferably from about 55 to about 75% by weight and in particular from about 58 to about 70% by weight, based on the total weight of the agent.

In one aspect, the agent is in the form of an aqueous-alcoholic solution.

In another aspect, the deodorizing cosmetic agent is propellant-free.

In yet another aspect, a pump spray contains the deodorizing cosmetic agent.

In one aspect, a roll-on contains the deodorizing cosmetic agent.

In another aspect, the deodorizing cosmetic agent is used to prevent and/or reduce body odor.

In yet another aspect, a non-therapeutic cosmetic method for preventing and/or reducing body odor includes applying the cosmetic agent according to skin, in particular to the skin of the underarms, and allowing the cosmetic agent to remain on the skin for at least about 1 hour, preferably for at least about 2 hours, more preferably for at least about 4 hours, in particular for at least about 6 hours.

The cosmetic agents according to embodiments of the present disclosure contain as their first essential constituent at least one quaternary ammonium compound. With regard to their technical properties, in particular their storage stability and cosmetic effect, cosmetic agents which contain as the quaternary ammonium compound at least one compound selected from the group consisting of behentrimonium chloride, cetrimonium bromide, cetrimonium chloride, laurtrimonium bromide, laurtrimonium chloride, steartrimonium bromide, steartrimonium chloride, benzethonium chloride and benzalkonium chloride have proven to be advantageous. The use of mixtures of the aforementioned quaternary ammonium compounds is of course also possible.

One quaternary ammonium compound which is particularly preferred from a technical point of view is benzalkonium chloride.

The proportion by weight of the quaternary ammonium compounds in the total weight of the cosmetic agent is preferably from about 0.01 to about 0.3% by weight, more preferably from about 0.02 to about 0.2% by weight and in particular from about 0.05 to about 0.1% by weight.

The second essential constituent of cosmetic agents according to an embodiment of the present disclosure is triethyl citrate. The proportion by weight of the triethyl citrate in the total weight of the cosmetic agent is preferably from about 0.1 to about 5.0% by weight, more preferably from about 0.25 to about 4.0% by weight and in particular from about 0.5 to about 3.0% by weight.

The cosmetic agents are in the form of an aqueous composition. Preferred cosmetic agents are characterized by a water content of from about 45 to about 85% by weight, preferably from about 55 to about 75% by weight and in particular from about 58 to about 70% by weight (in each case based on the total weight of the agent).

The cosmetic agents contain no aluminum-containing compounds. In the context of the present disclosure, antiperspirant aluminum salts and aluminum-zirconium salts will be understood to fall under the term “aluminum-containing compounds.”

Cosmetic deodorants counteract, mask or eliminate body odor. Body odor is caused by the action of skin bacteria on apocrine sweat, thereby forming decomposition products which smell unpleasant. Preferred deodorants therefore contain active ingredients which act as bacteriostatic agents, enzyme inhibitors, odor absorbers or odor masking agents.

Suitable bacteriostatic agents which may optionally be added to the cosmetic agents according to embodiments of the present disclosure are in principle all substances that are active against gram-positive bacteria, such as for example 4-hydroxybenzoic acid and salts and esters thereof, N-(4-chlorophenyl)-N′-(3,4-dichlorophenyl) urea, 2,4,4′-trichloro-2′-hydroxydiphenyl ether (triclosan), 4-chloro-3,5-dimethylphenol, 2,2′-methylene-bis(6-bromo-4-chlorophenol), 3-methyl-4-(1-methylethyl)phenol, 2-benzyl-4-chlorophenol, 3-(4-chlorophenoxy)-1,2-propanediol, 3-iodo-2-propynylbutyl carbamate, chlorhexidine, 3,4,4′-trichlorocarbanilide (TTC), antibacterial fragrances, thymol, thyme oil, eugenol, clove oil, mint oil, farnesol, phenoxyethanol, glycerol monolaurate (GML), diglycerol monocaprinate (DMC), salicylic acid N-alkylamides, such as for example salicylic acid n-octylamide or salicylic acid n-decylamide.

Enzyme inhibitors may be added to the cosmetic agents according to embodiments of the present disclosure, provided that they do not inhibit the enzyme activities that give rise to the biocide activation or biocide release in the presence of microorganisms to be combated. Under this condition, esterase inhibitors for example are enzyme inhibitors that may possibly be suitable. These are preferably trialkyl citrates such as trimethyl citrate, tripropyl citrate, triisopropyl citrate, tributyl citrate and in particular triethyl citrate (Hydagen® CAT, Henkel KgaA, Dusseldorf/Germany). The substances inhibit the enzyme activity and thus reduce the odor formation. Further substances suitable as esterase inhibitors are sterol sulfates or phosphates, such as for example lanosterol, cholesterol, campesterol, stigmasterol and sitosterol sulfate or phosphate, dicarboxylic acids and esters thereof, such as for example glutaric acid, monoethyl glutarate, diethyl glutarate, adipic acid, monoethyl adipate, diethyl adipate, malonic acid and diethyl malonate, hydroxycarboxylic acids and esters thereof, such as for example citric acid, malic acid, tartaric acid or diethyl tartrate, and zinc glycinate.

In combination with the active ingredient combination according to embodiments of the present disclosure, the use of bactericides has proven to be particularly advantageous. The use thereof, in particular the use of a bactericide from the group consisting of chlorhexidine, farnesol, triclosan, chloroxylenol and phenoxyethanol, preferably phenoxyethanol, unexpectedly had an advantageous effect on the odor development of the cosmetic agents. Preference is therefore given to deodorizing cosmetic agents which contain a bactericide from the group consisting of chlorhexidine, farnesol, triclosan, chloroxylenol and phenoxyethanol, preferably phenoxyethanol.

The proportion by weight of the bactericide, preferably of the phenoxyethanol, in the total weight of the cosmetic agent is preferably from about 0.1 to about 2.0% by weight, more preferably from about 0.2 to about 1.6% by weight and in particular from about 0.5 to about 1.4% by weight.

As a further preferred constituent, the agents according to embodiments of the present disclosure contain at least one emulsifier. Suitable emulsifiers are in principle anionic, cationic, nonionic and ampholytic surface-active compounds which are suitable for use on the human body. Ampholytic surface-active compounds include zwitterionic surface-active compounds and ampholytes. Preference is given to nonionic emulsifiers.

As nonionic emulsifiers, use can be made in particular of addition products of ethylene oxide with linear fatty alcohols, with fatty acids, with fatty acid alkanolamides, with fatty acid monoglycerides, with sorbitan fatty acid monoesters, with fatty acid glycerides, with methyl glucoside mono-fatty acid esters, with polydimethylsiloxanes, and mixtures thereof.

The emulsifier is preferably selected from the group consisting of addition products of about 2 to about 50 mol ethylene oxide with linear fatty alcohols having 12 to 18 carbon atoms and addition products of about 2 to about 50 mol propylene oxide with linear fatty alcohols having 12 to 18 carbon atoms. Examples of particularly preferred nonionic surfactants are compounds having the INCI names Ceteareth-12, Ceteareth-30 and in particular mixtures thereof.

Preferred deodorizing cosmetic agents contain the nonionic emulsifier in an amount of from about 1.0 to about 10% by weight, preferably from about 2.0 to about 8.0% by weight and in particular from about 3.0 to about 6.0% by weight, based on the total weight of the agent.

To thicken the cosmetic agents, use is preferably made of hydrogel-forming substances. Preferred thickening agents are selected from cellulose ethers, especially hydroxyalkyl celluloses, in particular hydroxypropyl cellulose, hydroxypropyl methyl cellulose, hydroxyethyl cellulose, carboxymethyl cellulose, cetyl hydroxyethyl cellulose, hydroxybutyl methyl cellulose and methyl hydroxyethyl cellulose. Further preferred thickening agents are xanthan gum, sclerotium gum, succinoglucans, polygalactomannans, in particular guar gums and locust bean gum, in particular guar gum and locust bean gum itself and the nonionic hydroxyalkyl guar derivatives and locust bean gum derivatives, such as hydroxypropyl guar, carboxymethyl hydroxypropyl guar, hydroxypropyl methyl guar, hydroxyethyl guar and carboxymethyl guar, also pectins, agar, carrageenan, tragacanth, gum arabic, karaya gum, tara gum, gellan, gelatin, casein, propylene glycol alginate, alginic acids and salts thereof, in particular sodium alginate, potassium alginate and calcium alginate, also polyvinylpyrrolidones, polyvinyl alcohols, polyacrylamides, also—although less preferred—physically modified (for example by pre-gelatinization) and/or chemically modified starches, in particular hydroxypropylated starch phosphates and octenyl starch succinates and the aluminum, calcium or sodium salts thereof, also—likewise less preferred—acrylic acid/acrylate copolymers, acrylic acid/acrylamide copolymers, acrylic acid/vinylpyrrolidone copolymers, acrylic acid/vinylformamide copolymers and polyacrylates.

Particularly preferred thickening agents are selected from cellulose ethers, in particular from the group consisting of hydroxypropyl cellulose, hydroxyethyl cellulose and methyl hydroxypropyl cellulose, in particular hydroxyethyl cellulose.

The proportion by weight of the cellulose ether, in particular hydroxyethyl cellulose, in the total weight of the cosmetic agent is preferably from about 0.1 to about 1.2% by weight, particularly preferably from about 0.15 to about 1.0% by weight and in particular from about 0.2 to about 0.8% by weight.

Preferred cosmetic agents are in the form of aqueous-alcoholic compositions, preferably in the form of aqueous-alcoholic solutions.

To improve the application properties, it is therefore preferred that the deodorizing cosmetic agents contain ethanol in amounts between about 10 and about 50% by weight, preferably between about 20 and about 40% by weight and in particular between about 23 and about 35% by weight, based on their total weight.

Besides the ingredients described above, the deodorizing cosmetic agents may contain further active ingredients and auxiliaries. The group of said further active ingredients and auxiliaries includes for example fragrances.

Particularly preferred cosmetic agents according to embodiments of the present disclosure further contain at least one fragrance. The term fragrance denotes a chemical compound having a smell and/or taste which stimulates the receptors of the hair cells of the olfactory system (adequate stimulus). The physical and chemical properties necessary for this are a low molar mass of at most about 300 g/mol, a high vapor pressure, minimal water solubility and high lipid solubility, weak polarity, and the presence of at least one osmophoric group in the molecule. In order to delineate volatile, low-molecular-weight substances, which usually and also in the context of the present application are regarded and used not as a fragrance but primarily as a solvent, such as for example ethanol, propanol, isopropanol and acetone, from fragrances, fragrances according to embodiments of the present disclosure have a molar mass of from about 74 to about 300 g/mol, contain at least one osmophoric group in the molecule and have a smell and/or taste, that is to say they stimulate the receptors of the hair cells of the olfactory system.

As fragrances, use can be made of perfumes, perfume oils or perfume oil constituents. According to various embodiments of the present disclosure, perfume oils or scents may be individual fragrance compounds, for example synthetic products of the ester, ether, aldehyde, ketone, alcohol and hydrocarbon type. Fragrance compounds of the ester type are for example benzyl acetate, phenoxyethyl isobutyrate, p-tert-butyl cyclohexyl acetate, linalyl acetate, dimethyl benzyl carbinyl acetate (DMBCA), phenylethyl acetate, benzyl acetate, ethyl methyl phenyl glycinate, allyl cyclohexyl propionate, styrallyl propionate, benzyl salicylate, cyclohexyl salicylate, floramat, melusat and jasmecyclat. The ethers include for example benzyl ethyl ether and Ambroxan; the aldehydes include for example the linear alkanals having 8-18 C atoms, citral, citronellal, citronellyl oxyacetaldehyde, cyclamen aldehyde, lilial and bourgeonal; the ketones include for example the ionones, alpha-isomethyl ionone and methyl cedryl ketone; the alcohols include anethol, citronellol, eugenol, geraniol, linalool, phenylethyl alcohol and terpineol; and the hydrocarbons include primarily the terpenes such as limonene and pinene. Preferably, however, use will be made of mixtures of different fragrances which together produce a pleasant scent note. Preferred cosmetic agents according to various embodiments of the present disclosure contain at least one fragrance component in a total amount of from about 0.00001 to about 10% by weight, preferably from about 0.5 to about 5% by weight, extremely preferably from about 1 to about 4% by weight, in each case based on the total weight of the agent.

The composition of some preferred cosmetic agents can be seen in the following tables (unless indicated otherwise, figures specified in % by weight are based on the total weight of the cosmetic agent).

| | Formula 1 | Formula 2 | Formula 3 | Formula 4 | Formula 5 |
|---|---|---|---|---|---|
| Quaternary ammonium | 0.01 to 0.3 | 0.01 to 0.3 | 0.02 to 0.2 | 0.02 to 0.2 | 0.05 to 0.1 |
| compound | | | | | |
| Triethyl citrate | 0.1 to 5.0 | 0.25 to 4.0 | 0.25 to 4.0 | 0.5 to 3.0 | 0.5 to 3.0 |
| Water, optional additives | ad 100 | ad 100 | ad 100 | ad 100 | ad 100 |

| | Formula 6 | Formula 7 | Formula 8 | Formula 9 | Formula 10 |
|---|---|---|---|---|---|
| Benzalkonium chloride | 0.01 to 0.3 | 0.01 to 0.3 | 0.02 to 0.2 | 0.02 to 0.2 | 0.05 to 0.1 |
| Triethyl citrate | 0.1 to 5.0 | 0.25 to 4.0 | 0.25 to 4.0 | 0.5 to 3.0 | 0.5 to 3.0 |
| Water, optional additives | ad 100 | ad 100 | ad 100 | ad 100 | ad 100 |

| | Formula 11 | Formula 12 | Formula 13 | Formula 14 | Formula 15 |
|---|---|---|---|---|---|
| Quaternary ammonium | 0.01 to 0.3 | 0.01 to 0.3 | 0.02 to 0.2 | 0.02 to 0.2 | 0.05 to 0.1 |
| compound | | | | | |
| Triethyl citrate | 0.1 to 5.0 | 0.25 to 4.0 | 0.25 to 4.0 | 0.5 to 3.0 | 0.5 to 3.0 |
| Bactericide | 0.1 to 2.0 | 0.1 to 2.0 | 0.2 to 1.6 | 0.2 to 1.6 | 0.5 to 1.4 |
| Water, optional additives | ad 100 | ad 100 | ad 100 | ad 100 | ad 100 |

| | Formula 16 | Formula 17 | Formula 18 | Formula 19 | Formula 20 |
|---|---|---|---|---|---|
| Benzalkonium chloride | 0.01 to 0.3 | 0.01 to 0.3 | 0.02 to 0.2 | 0.02 to 0.2 | 0.05 to 0.1 |
| Triethyl citrate | 0.1 to 5.0 | 0.25 to 4.0 | 0.25 to 4.0 | 0.5 to 3.0 | 0.5 to 3.0 |
| Bactericide | 0.1 to 2.0 | 0.1 to 2.0 | 0.2 to 1.6 | 0.2 to 1.6 | 0.5 to 1.4 |
| Water, optional additives | ad 100 | ad 100 | ad 100 | ad 100 | ad 100 |

| | Formula 21 | Formula 22 | Formula 23 | Formula 24 | Formula 25 |
|---|---|---|---|---|---|
| Quaternary ammonium | 0.01 to 0.3 | 0.01 to 0.3 | 0.02 to 0.2 | 0.02 to 0.2 | 0.05 to 0.1 |
| compound | | | | | |
| Triethyl citrate | 0.1 to 5.0 | 0.25 to 4.0 | 0.25 to 4.0 | 0.5 to 3.0 | 0.5 to 3.0 |
| Phenoxyethanol | 0.1 to 2.0 | 0.1 to 2.0 | 0.2 to 1.6 | 0.2 to 1.6 | 0.5 to 1.4 |
| Water, optional additives | ad 100 | ad 100 | ad 100 | ad 100 | ad 100 |

| | Formula 26 | Formula 27 | Formula 28 | Formula 29 | Formula 30 |
|---|---|---|---|---|---|
| Benzalkonium chloride | 0.01 to 0.3 | 0.01 to 0.3 | 0.02 to 0.2 | 0.02 to 0.2 | 0.05 to 0.1 |
| Triethyl citrate | 0.1 to 5.0 | 0.25 to 4.0 | 0.25 to 4.0 | 0.5 to 3.0 | 0.5 to 3.0 |
| Phenoxyethanol | 0.1 to 2.0 | 0.1 to 2.0 | 0.2 to 1.6 | 0.2 to 1.6 | 0.5 to 1.4 |
| Water, optional additives | ad 100 | ad 100 | ad 100 | ad 100 | ad 100 |

| | Formula | Formula | Formula | Formula | |
|---|---|---|---|---|---|
| | 31 | 32 | 33 | 34 | Formula 35 |
| Quaternary ammonium | 0.01 to 0.3 | 0.01 to 0.3 | 0.02 to 0.2 | 0.02 to 0.2 | 0.05 to 0.1 |
| compound | | | | | |
| Triethyl citrate | 0.1 to 5.0 | 0.25 to 4.0 | 0.25 to 4.0 | 0.5 to 3.0 | 0.5 to 3.0 |
| Nonionic emulsifier | 3.0 to 12 | 3.0 to 12 | 4.0 to 10 | 4.0 to 10 | 5.0 to 8.0 |
| Water, optional additives | ad 100 | ad 100 | ad 100 | ad 100 | ad 100 |

| | Formula | Formula | Formula | Formula | |
|---|---|---|---|---|---|
| | 36 | 37 | 38 | 39 | Formula 40 |
| Benzalkonium chloride | 0.01 to 0.3 | 0.01 to 0.3 | 0.02 to 0.2 | 0.02 to 0.2 | 0.05 to 0.1 |
| Triethyl citrate | 0.1 to 5.0 | 0.25 to 4.0 | 0.25 to 4.0 | 0.5 to 3.0 | 0.5 to 3.0 |
| Nonionic emulsifier | 3.0 to 12 | 3.0 to 12 | 4.0 to 10 | 4.0 to 10 | 5.0 to 8.0 |
| Water, optional additives | ad 100 | ad 100 | ad 100 | ad 100 | ad 100 |

| | Formula | Formula | Formula | Formula | |
|---|---|---|---|---|---|
| | 41 | 42 | 43 | 44 | Formula 45 |
| Quaternary ammonium | 0.01 to 0.3 | 0.01 to 0.3 | 0.02 to 0.2 | 0.02 to 0.2 | 0.05 to 0.1 |
| compound | | | | | |
| Triethyl citrate | 0.1 to 5.0 | 0.25 to 4.0 | 0.25 to 4.0 | 0.5 to 3.0 | 0.5 to 3.0 |
| Nonionic emulsifier* | 3.0 to 12 | 3.0 to 12 | 4.0 to 10 | 4.0 to 10 | 5.0 to 8.0 |
| Water, optional additives | ad 100 | ad 100 | ad 100 | ad 100 | ad 100 |

*Ceteareth-12, Ceteareth-30 or combinations thereof

| | Formula | Formula | Formula | Formula | |
|---|---|---|---|---|---|
| | 46 | 47 | 48 | 49 | Formula 50 |
| Benzalkonium chloride | 0.01 to 0.3 | 0.01 to 0.3 | 0.02 to 0.2 | 0.02 to 0.2 | 0.05 to 0.1 |
| Triethyl citrate | 0.1 to 5.0 | 0.25 to 4.0 | 0.25 to 4.0 | 0.5 to 3.0 | 0.5 to 3.0 |
| Nonionic emulsifier* | 3.0 to 12 | 3.0 to 12 | 4.0 to 10 | 4.0 to 10 | 5.0 to 8.0 |
| Water, optional additives | ad 100 | ad 100 | ad 100 | ad 100 | ad 100 |

*Ceteareth-12, Ceteareth-30 or combinations thereof

| | Formula | Formula | Formula | Formula | |
|---|---|---|---|---|---|
| | 51 | 52 | 53 | 54 | Formula 55 |
| Quaternary ammonium | 0.01 to 0.3 | 0.01 to 0.3 | 0.02 to 0.2 | 0.02 to 0.2 | 0.05 to 0.1 |
| compound | | | | | |
| Triethyl citrate | 0.1 to 5.0 | 0.25 to 4.0 | 0.25 to 4.0 | 0.5 to 3.0 | 0.5 to 3.0 |
| Bactericide | 0.1 to 2.0 | 0.1 to 2.0 | 0.2 to 1.6 | 0.2 to 1.6 | 0.5 to 1.4 |
| Nonionic emulsifier | 3.0 to 12 | 3.0 to 12 | 4.0 to 10 | 4.0 to 10 | 5.0 to 8.0 |
| Water, optional additives | ad 100 | ad 100 | ad 100 | ad 100 | ad 100 |

| | Formula | Formula | Formula | Formula | |
|---|---|---|---|---|---|
| | 56 | 57 | 58 | 59 | Formula 60 |
| Benzalkonium chloride | 0.01 to 0.3 | 0.01 to 0.3 | 0.02 to 0.2 | 0.02 to 0.2 | 0.05 to 0.1 |
| Triethyl citrate | 0.1 to 5.0 | 0.25 to 4.0 | 0.25 to 4.0 | 0.5 to 3.0 | 0.5 to 3.0 |
| Bactericide | 0.1 to 2.0 | 0.1 to 2.0 | 0.2 to 1.6 | 0.2 to 1.6 | 0.5 to 1.4 |
| Nonionic emulsifier | 3.0 to 12 | 3.0 to 12 | 4.0 to 10 | 4.0 to 10 | 5.0 to 8.0 |
| Water, optional additives | ad 100 | ad 100 | ad 100 | ad 100 | ad 100 |

| | Formula | Formula | Formula | Formula | |
|---|---|---|---|---|---|
| | 61 | 62 | 63 | 64 | Formula 65 |
| Quaternary ammonium | 0.01 to 0.3 | 0.01 to 0.3 | 0.02 to 0.2 | 0.02 to 0.2 | 0.05 to 0.1 |
| compound | | | | | |
| Triethyl citrate | 0.1 to 5.0 | 0.25 to 4.0 | 0.25 to 4.0 | 0.5 to 3.0 | 0.5 to 3.0 |
| Bactericide | 0.1 to 2.0 | 0.1 to 2.0 | 0.2 to 1.6 | 0.2 to 1.6 | 0.5 to 1.4 |
| Nonionic emulsifier* | 3.0 to 12 | 3.0 to 12 | 4.0 to 10 | 4.0 to 10 | 5.0 to 8.0 |
| Water, optional additives | ad 100 | ad 100 | ad 100 | ad 100 | ad 100 |

*Ceteareth-12, Ceteareth-30 or combinations thereof

| | Formula | Formula | Formula | Formula | |
|---|---|---|---|---|---|
| | 66 | 67 | 68 | 69 | Formula 70 |
| Benzalkonium chloride | 0.01 to 0.3 | 0.01 to 0.3 | 0.02 to 0.2 | 0.02 to 0.2 | 0.05 to 0.1 |
| Triethyl citrate | 0.1 to 5.0 | 0.25 to 4.0 | 0.25 to 4.0 | 0.5 to 3.0 | 0.5 to 3.0 |
| Bactericide | 0.1 to 2.0 | 0.1 to 2.0 | 0.2 to 1.6 | 0.2 to 1.6 | 0.5 to 1.4 |
| Nonionic emulsifier* | 3.0 to 12 | 3.0 to 12 | 4.0 to 10 | 4.0 to 10 | 5.0 to 8.0 |
| Water, optional additives | ad 100 | ad 100 | ad 100 | ad 100 | ad 100 |

*Ceteareth-12, Ceteareth-30 or combinations thereof

| | Formula | Formula | Formula | Formula | |
|---|---|---|---|---|---|
| | 71 | 72 | 73 | 74 | Formula 75 |
| Quaternary ammonium | 0.01 to 0.3 | 0.01 to 0.3 | 0.02 to 0.2 | 0.02 to 0.2 | 0.05 to 0.1 |
| compound | | | | | |
| Triethyl citrate | 0.1 to 5.0 | 0.25 to 4.0 | 0.25 to 4.0 | 0.5 to 3.0 | 0.5 to 3.0 |
| Phenoxyethanol | 0.1 to 2.0 | 0.1 to 2.0 | 0.2 to 1.6 | 0.2 to 1.6 | 0.5 to 1.4 |
| Nonionic emulsifier | 3.0 to 12 | 3.0 to 12 | 4.0 to 10 | 4.0 to 10 | 5.0 to 8.0 |
| Water, optional additives | ad 100 | ad 100 | ad 100 | ad 100 | ad 100 |

| | Formula | Formula | Formula | Formula | |
|---|---|---|---|---|---|
| | 76 | 77 | 78 | 79 | Formula 80 |
| Benzalkonium chloride | 0.01 to 0.3 | 0.01 to 0.3 | 0.02 to 0.2 | 0.02 to 0.2 | 0.05 to 0.1 |
| Triethyl citrate | 0.1 to 5.0 | 0.25 to 4.0 | 0.25 to 4.0 | 0.5 to 3.0 | 0.5 to 3.0 |
| Phenoxyethanol | 0.1 to 2.0 | 0.1 to 2.0 | 0.2 to 1.6 | 0.2 to 1.6 | 0.5 to 1.4 |
| Nonionic emulsifier | 3.0 to 12 | 3.0 to 12 | 4.0 to 10 | 4.0 to 10 | 5.0 to 8.0 |
| Water, optional additives | ad 100 | ad 100 | ad 100 | ad 100 | ad 100 |

| | Formula | Formula | Formula | Formula | |
|---|---|---|---|---|---|
| | 81 | 82 | 83 | 84 | Formula 85 |
| Quaternary ammonium | 0.01 to 0.3 | 0.01 to 0.3 | 0.02 to 0.2 | 0.02 to 0.2 | 0.05 to 0.1 |
| compound | | | | | |
| Triethyl citrate | 0.1 to 5.0 | 0.25 to 4.0 | 0.25 to 4.0 | 0.5 to 3.0 | 0.5 to 3.0 |
| Phenoxyethanol | 0.1 to 2.0 | 0.1 to 2.0 | 0.2 to 1.6 | 0.2 to 1.6 | 0.5 to 1.4 |
| Nonionic emulsifier* | 3.0 to 12 | 3.0 to 12 | 4.0 to 10 | 4.0 to 10 | 5.0 to 8.0 |
| Water, optional additives | ad 100 | ad 100 | ad 100 | ad 100 | ad 100 |

*Ceteareth-12, Ceteareth-30 or combinations thereof

| | Formula | Formula | Formula | Formula | |
|---|---|---|---|---|---|
| | 86 | 87 | 88 | 89 | Formula 90 |
| Benzalkonium chloride | 0.01 to 0.3 | 0.01 to 0.3 | 0.02 to 0.2 | 0.02 to 0.2 | 0.05 to 0.1 |
| Triethyl citrate | 0.1 to 5.0 | 0.25 to 4.0 | 0.25 to 4.0 | 0.5 to 3.0 | 0.5 to 3.0 |
| Phenoxyethanol | 0.1 to 2.0 | 0.1 to 2.0 | 0.2 to 1.6 | 0.2 to 1.6 | 0.5 to 1.4 |
| Nonionic emulsifier* | 3.0 to 12 | 3.0 to 12 | 4.0 to 10 | 4.0 to 10 | 5.0 to 8.0 |
| Water, optional additives | ad 100 | ad 100 | ad 100 | ad 100 | ad 100 |

*Ceteareth-12, Ceteareth-30 or combinations thereof

| | Formula | Formula | Formula | Formula | Formula |
|---|---|---|---|---|---|
| | 101 | 102 | 103 | 104 | 105 |
| Quaternary ammonium | 0.01 to 0.3 | 0.01 to 0.3 | 0.02 to 0.2 | 0.02 to 0.2 | 0.05 to 0.1 |
| compound | | | | | |
| Triethyl citrate | 0.1 to 5.0 | 0.25 to 4.0 | 0.25 to 4.0 | 0.5 to 3.0 | 0.5 to 3.0 |
| Ethanol | 10 to 50 | 10 to 50 | 20 to 40 | 20 to 40 | 23 to 35 |
| Water | 45 to 85 | 45 to 85 | 55 to 75 | 55 to 75 | 58 to 70 |
| Optional additives | ad 100 | ad 100 | ad 100 | ad 100 | ad 100 |

| | Formula | Formula | Formula | Formula | Formula |
|---|---|---|---|---|---|
| | 106 | 107 | 108 | 109 | 110 |
| Benzalkonium chloride | 0.01 to 0.3 | 0.01 to 0.3 | 0.02 to 0.2 | 0.02 to 0.2 | 0.05 to 0.1 |
| Triethyl citrate | 0.1 to 5.0 | 0.25 to 4.0 | 0.25 to 4.0 | 0.5 to 3.0 | 0.5 to 3.0 |
| Ethanol | 10 to 50 | 10 to 50 | 20 to 40 | 20 to 40 | 23 to 35 |
| Water | 45 to 85 | 45 to 85 | 55 to 75 | 55 to 75 | 58 to 70 |
| Optional additives | ad 100 | ad 100 | ad 100 | ad 100 | ad 100 |

| | Formula | Formula | Formula | Formula | Formula |
|---|---|---|---|---|---|
| | 111 | 112 | 113 | 114 | 115 |
| Quaternary ammonium | 0.01 to 0.3 | 0.01 to 0.3 | 0.02 to 0.2 | 0.02 to 0.2 | 0.05 to 0.1 |
| compound | | | | | |
| Triethyl citrate | 0.1 to 5.0 | 0.25 to 4.0 | 0.25 to 4.0 | 0.5 to 3.0 | 0.5 to 3.0 |
| Bactericide | 0.1 to 2.0 | 0.1 to 2.0 | 0.2 to 1.6 | 0.2 to 1.6 | 0.5 to 1.4 |
| Ethanol | 10 to 50 | 10 to 50 | 20 to 40 | 20 to 40 | 23 to 35 |
| Water | 45 to 85 | 45 to 85 | 55 to 75 | 55 to 75 | 58 to 70 |
| Optional additives | ad 100 | ad 100 | ad 100 | ad 100 | ad 100 |

| | Formula 116 | Formula 117 | Formula 118 | Formula 119 | Formula 120 |
|---|---|---|---|---|---|
| Benzalkonium chloride | 0.01 to 0.3 | 0.01 to 0.3 | 0.02 to 0.2 | 0.02 to 0.2 | 0.05 to 0.1 |
| Triethyl citrate | 0.1 to 5.0 | 0.25 to 4.0 | 0.25 to 4.0 | 0.5 to 3.0 | 0.5 to 3.0 |
| Bactericide | 0.1 to 2.0 | 0.1 to 2.0 | 0.2 to 1.6 | 0.2 to 1.6 | 0.5 to 1.4 |
| Ethanol | 10 to 50 | 10 to 50 | 20 to 40 | 20 to 40 | 23 to 35 |
| Water | 45 to 85 | 45 to 85 | 55 to 75 | 55 to 75 | 58 to 70 |
| Optional additives | ad 100 | ad 100 | ad 100 | ad 100 | ad 100 |

| | Formula 121 | Formula 122 | Formula 123 | Formula 124 | Formula 125 |
|---|---|---|---|---|---|
| Quaternary ammonium | 0.01 to 0.3 | 0.01 to 0.3 | 0.02 to 0.2 | 0.02 to 0.2 | 0.05 to 0.1 |
| compound | | | | | |
| Triethyl citrate | 0.1 to 5.0 | 0.25 to 4.0 | 0.25 to 4.0 | 0.5 to 3.0 | 0.5 to 3.0 |
| Phenoxyethanol | 0.1 to 2.0 | 0.1 to 2.0 | 0.2 to 1.6 | 0.2 to 1.6 | 0.5 to 1.4 |
| Ethanol | 10 to 50 | 10 to 50 | 20 to 40 | 20 to 40 | 23 to 35 |
| Water | 45 to 85 | 45 to 85 | 55 to 75 | 55 to 75 | 58 to 70 |
| Optional additives | ad 100 | ad 100 | ad 100 | ad 100 | ad 100 |

| | Formula 126 | Formula 127 | Formula 128 | Formula 129 | Formula 130 |
|---|---|---|---|---|---|
| Benzalkonium chloride | 0.01 to 0.3 | 0.01 to 0.3 | 0.02 to 0.2 | 0.02 to 0.2 | 0.05 to 0.1 |
| Triethyl citrate | 0.1 to 5.0 | 0.25 to 4.0 | 0.25 to 4.0 | 0.5 to 3.0 | 0.5 to 3.0 |
| Phenoxyethanol | 0.1 to 2.0 | 0.1 to 2.0 | 0.2 to 1.6 | 0.2 to 1.6 | 0.5 to 1.4 |
| Ethanol | 10 to 50 | 10 to 50 | 20 to 40 | 20 to 40 | 23 to 35 |
| Water | 45 to 85 | 45 to 85 | 55 to 75 | 55 to 75 | 58 to 70 |
| Optional additives | ad 100 | ad 100 | ad 100 | ad 100 | ad 100 |

| | Formula 131 | Formula 132 | Formula 133 | Formula 134 | Formula 135 |
|---|---|---|---|---|---|
| Quaternary ammonium | 0.01 to 0.3 | 0.01 to 0.3 | 0.02 to 0.2 | 0.02 to 0.2 | 0.05 to 0.1 |
| compound | | | | | |
| Triethyl citrate | 0.1 to 5.0 | 0.25 to 4.0 | 0.25 to 4.0 | 0.5 to 3.0 | 0.5 to 3.0 |
| Nonionic emulsifier | 3.0 to 12 | 3.0 to 12 | 4.0 to 10 | 4.0 to 10 | 5.0 to 8.0 |
| Ethanol | 10 to 50 | 10 to 50 | 20 to 40 | 20 to 40 | 23 to 35 |
| Water | 45 to 85 | 45 to 85 | 55 to 75 | 55 to 75 | 58 to 70 |
| Optional additives | ad 100 | ad 100 | ad 100 | ad 100 | ad 100 |

| | Formula 136 | Formula 137 | Formula 138 | Formula 139 | Formula 140 |
|---|---|---|---|---|---|
| Benzalkonium chloride | 0.01 to 0.3 | 0.01 to 0.3 | 0.02 to 0.2 | 0.02 to 0.2 | 0.05 to 0.1 |
| Triethyl citrate | 0.1 to 5.0 | 0.25 to 4.0 | 0.25 to 4.0 | 0.5 to 3.0 | 0.5 to 3.0 |
| Nonionic emulsifier | 3.0 to 12 | 3.0 to 12 | 4.0 to 10 | 4.0 to 10 | 5.0 to 8.0 |
| Ethanol | 10 to 50 | 10 to 50 | 20 to 40 | 20 to 40 | 23 to 35 |
| Water | 45 to 85 | 45 to 85 | 55 to 75 | 55 to 75 | 58 to 70 |
| Optional additives | ad 100 | ad 100 | ad 100 | ad 100 | ad 100 |

| | Formula 141 | Formula 142 | Formula 143 | Formula 144 | Formula 145 |
|---|---|---|---|---|---|
| Quaternary ammonium | 0.01 to 0.3 | 0.01 to 0.3 | 0.02 to 0.2 | 0.02 to 0.2 | 0.05 to 0.1 |
| compound | | | | | |
| Triethyl citrate | 0.1 to 5.0 | 0.25 to 4.0 | 0.25 to 4.0 | 0.5 to 3.0 | 0.5 to 3.0 |
| Nonionic emulsifier* | 3.0 to 12 | 3.0 to 12 | 4.0 to 10 | 4.0 to 10 | 5.0 to 8.0 |
| Ethanol | 10 to 50 | 10 to 50 | 20 to 40 | 20 to 40 | 23 to 35 |
| Water | 45 to 85 | 45 to 85 | 55 to 75 | 55 to 75 | 58 to 70 |
| Optional additives | ad 100 | ad 100 | ad 100 | ad 100 | ad 100 |

*Ceteareth-12, Ceteareth-30 or combinations thereof

| | Formula 146 | Formula 147 | Formula 148 | Formula 149 | Formula 150 |
|---|---|---|---|---|---|
| Benzalkonium chloride | 0.01 to 0.3 | 0.01 to 0.3 | 0.02 to 0.2 | 0.02 to 0.2 | 0.05 to 0.1 |
| Triethyl citrate | 0.1 to 5.0 | 0.25 to 4.0 | 0.25 to 4.0 | 0.5 to 3.0 | 0.5 to 3.0 |
| Nonionic emulsifier* | 3.0 to 12 | 3.0 to 12 | 4.0 to 10 | 4.0 to 10 | 5.0 to 8.0 |
| Ethanol | 10 to 50 | 10 to 50 | 20 to 40 | 20 to 40 | 23 to 35 |
| Water | 45 to 85 | 45 to 85 | 55 to 75 | 55 to 75 | 58 to 70 |
| Optional additives | ad 100 | ad 100 | ad 100 | ad 100 | ad 100 |

*Ceteareth-12, Ceteareth-30 or combinations thereof

| | Formula 151 | Formula 152 | Formula 153 | Formula 154 | Formula 155 |
|---|---|---|---|---|---|
| Quaternary ammonium | 0.01 to 0.3 | 0.01 to 0.3 | 0.02 to 0.2 | 0.02 to 0.2 | 0.05 to 0.1 |
| compound | | | | | |
| Triethyl citrate | 0.1 to 5.0 | 0.25 to 4.0 | 0.25 to 4.0 | 0.5 to 3.0 | 0.5 to 3.0 |
| Bactericide | 0.1 to 2.0 | 0.1 to 2.0 | 0.2 to 1.6 | 0.2 to 1.6 | 0.5 to 1.4 |
| Nonionic emulsifier | 3.0 to 12 | 3.0 to 12 | 4.0 to 10 | 4.0 to 10 | 5.0 to 8.0 |
| Ethanol | 10 to 50 | 10 to 50 | 20 to 40 | 20 to 40 | 23 to 35 |
| Water | 45 to 85 | 45 to 85 | 55 to 75 | 55 to 75 | 58 to 70 |
| Optional additives | ad 100 | ad 100 | ad 100 | ad 100 | ad 100 |

| | Formula 156 | Formula 157 | Formula 158 | Formula 159 | Formula 160 |
|---|---|---|---|---|---|
| Benzalkonium chloride | 0.01 to 0.3 | 0.01 to 0.3 | 0.02 to 0.2 | 0.02 to 0.2 | 0.05 to 0.1 |
| Triethyl citrate | 0.1 to 5.0 | 0.25 to 4.0 | 0.25 to 4.0 | 0.5 to 3.0 | 0.5 to 3.0 |
| Bactericide | 0.1 to 2.0 | 0.1 to 2.0 | 0.2 to 1.6 | 0.2 to 1.6 | 0.5 to 1.4 |
| Nonionic emulsifier | 3.0 to 12 | 3.0 to 12 | 4.0 to 10 | 4.0 to 10 | 5.0 to 8.0 |
| Ethanol | 10 to 50 | 10 to 50 | 20 to 40 | 20 to 40 | 23 to 35 |
| Water | 45 to 85 | 45 to 85 | 55 to 75 | 55 to 75 | 58 to 70 |
| Optional additives | ad 100 | ad 100 | ad 100 | ad 100 | ad 100 |

| | Formula 161 | Formula 162 | Formula 163 | Formula 164 | Formula 165 |
|---|---|---|---|---|---|
| Quaternary ammonium | 0.01 to 0.3 | 0.01 to 0.3 | 0.02 to 0.2 | 0.02 to 0.2 | 0.05 to 0.1 |
| compound | | | | | |
| Triethyl citrate | 0.1 to 5.0 | 0.25 to 4.0 | 0.25 to 4.0 | 0.5 to 3.0 | 0.5 to 3.0 |
| Bactericide | 0.1 to 2.0 | 0.1 to 2.0 | 0.2 to 1.6 | 0.2 to 1.6 | 0.5 to 1.4 |
| Nonionic emulsifier* | 3.0 to 12 | 3.0 to 12 | 4.0 to 10 | 4.0 to 10 | 5.0 to 8.0 |
| Ethanol | 10 to 50 | 10 to 50 | 20 to 40 | 20 to 40 | 23 to 35 |
| Water | 45 to 85 | 45 to 85 | 55 to 75 | 55 to 75 | 58 to 70 |
| Optional additives | ad 100 | ad 100 | ad 100 | ad 100 | ad 100 |

*Ceteareth-12, Ceteareth-30 or combinations thereof

| | Formula 166 | Formula 167 | Formula 168 | Formula 169 | Formula 170 |
|---|---|---|---|---|---|
| Benzalkonium chloride | 0.01 to 0.3 | 0.01 to 0.3 | 0.02 to 0.2 | 0.02 to 0.2 | 0.05 to 0.1 |
| Triethyl citrate | 0.1 to 5.0 | 0.25 to 4.0 | 0.25 to 4.0 | 0.5 to 3.0 | 0.5 to 3.0 |
| Bactericide | 0.1 to 2.0 | 0.1 to 2.0 | 0.2 to 1.6 | 0.2 to 1.6 | 0.5 to 1.4 |
| Nonionic emulsifier* | 3.0 to 12 | 3.0 to 12 | 4.0 to 10 | 4.0 to 10 | 5.0 to 8.0 |
| Ethanol | 10 to 50 | 10 to 50 | 20 to 40 | 20 to 40 | 23 to 35 |
| Water | 45 to 85 | 45 to 85 | 55 to 75 | 55 to 75 | 58 to 70 |
| Optional additives | ad 100 | ad 100 | ad 100 | ad 100 | ad 100 |

*Ceteareth-12, Ceteareth-30 or combinations thereof

| | Formula 171 | Formula 172 | Formula 173 | Formula 174 | Formula 175 |
|---|---|---|---|---|---|
| Quaternary ammonium | 0.01 to 0.3 | 0.01 to 0.3 | 0.02 to 0.2 | 0.02 to 0.2 | 0.05 to 0.1 |
| compound | | | | | |
| Triethyl citrate | 0.1 to 5.0 | 0.25 to 4.0 | 0.25 to 4.0 | 0.5 to 3.0 | 0.5 to 3.0 |
| Phenoxyethanol | 0.1 to 2.0 | 0.1 to 2.0 | 0.2 to 1.6 | 0.2 to 1.6 | 0.5 to 1.4 |
| Nonionic emulsifier | 3.0 to 12 | 3.0 to 12 | 4.0 to 10 | 4.0 to 10 | 5.0 to 8.0 |
| Ethanol | 10 to 50 | 10 to 50 | 20 to 40 | 20 to 40 | 23 to 35 |
| Water | 45 to 85 | 45 to 85 | 55 to 75 | 55 to 75 | 58 to 70 |
| Optional additives | ad 100 | ad 100 | ad 100 | ad 100 | ad 100 |

| | Formula 176 | Formula 177 | Formula 178 | Formula 179 | Formula 180 |
|---|---|---|---|---|---|
| Benzalkonium chloride | 0.01 to 0.3 | 0.01 to 0.3 | 0.02 to 0.2 | 0.02 to 0.2 | 0.05 to 0.1 |
| Triethyl citrate | 0.1 to 5.0 | 0.25 to 4.0 | 0.25 to 4.0 | 0.5 to 3.0 | 0.5 to 3.0 |
| Phenoxyethanol | 0.1 to 2.0 | 0.1 to 2.0 | 0.2 to 1.6 | 0.2 to 1.6 | 0.5 to 1.4 |
| Nonionic emulsifier | 3.0 to 12 | 3.0 to 12 | 4.0 to 10 | 4.0 to 10 | 5.0 to 8.0 |
| Ethanol | 10 to 50 | 10 to 50 | 20 to 40 | 20 to 40 | 23 to 35 |
| Water | 45 to 85 | 45 to 85 | 55 to 75 | 55 to 75 | 58 to 70 |
| Optional additives | ad 100 | ad 100 | ad 100 | ad 100 | ad 100 |

| | Formula 181 | Formula 182 | Formula 183 | Formula 184 | Formula 185 |
|---|---|---|---|---|---|
| Quaternary ammonium | 0.01 to 0.3 | 0.01 to 0.3 | 0.02 to 0.2 | 0.02 to 0.2 | 0.05 to 0.1 |
| compound | | | | | |
| Triethyl citrate | 0.1 to 5.0 | 0.25 to 4.0 | 0.25 to 4.0 | 0.5 to 3.0 | 0.5 to 3.0 |
| Phenoxyethanol | 0.1 to 2.0 | 0.1 to 2.0 | 0.2 to 1.6 | 0.2 to 1.6 | 0.5 to 1.4 |
| Nonionic emulsifier* | 3.0 to 12 | 3.0 to 12 | 4.0 to 10 | 4.0 to 10 | 5.0 to 8.0 |
| Ethanol | 10 to 50 | 10 to 50 | 20 to 40 | 20 to 40 | 23 to 35 |
| Water | 45 to 85 | 45 to 85 | 55 to 75 | 55 to 75 | 58 to 70 |
| Optional additives | ad 100 | ad 100 | ad 100 | ad 100 | ad 100 |

*Ceteareth-12, Ceteareth-30 or combinations thereof

| | Formula 186 | Formula 187 | Formula 188 | Formula 189 | Formula 190 |
|---|---|---|---|---|---|
| Benzalkonium chloride | 0.01 to 0.3 | 0.01 to 0.3 | 0.02 to 0.2 | 0.02 to 0.2 | 0.05 to 0.1 |
| Triethyl citrate | 0.1 to 5.0 | 0.25 to 4.0 | 0.25 to 4.0 | 0.5 to 3.0 | 0.5 to 3.0 |
| Phenoxyethanol | 0.1 to 2.0 | 0.1 to 2.0 | 0.2 to 1.6 | 0.2 to 1.6 | 0.5 to 1.4 |
| Nonionic emulsifier* | 3.0 to 12 | 3.0 to 12 | 4.0 to 10 | 4.0 to 10 | 5.0 to 8.0 |
| Ethanol | 10 to 50 | 10 to 50 | 20 to 40 | 20 to 40 | 23 to 35 |
| Water | 45 to 85 | 45 to 85 | 55 to 75 | 55 to 75 | 58 to 70 |
| Optional additives | ad 100 | ad 100 | ad 100 | ad 100 | ad 100 |

*Ceteareth-12, Ceteareth-30 or combinations thereof

The deodorizing cosmetic agent may be applied by various methods. According to one preferred embodiment, the antiperspirant cosmetic agent is prepared as a spray application. The spray application takes place by means of a spray device which contains in a container a filling consisting of the deodorizing cosmetic agent. The filling may be under the pressure of a propellant (pressurized gas cans, pressurized gas packagings, aerosol packagings), or it may be a mechanically operated pump atomizer without propellant gas (pump sprays/squeeze bottles). Propellant gas is preferably omitted, and preferred agents are therefore propellant-free. The resulting preferred propellant-free deodorizing agents are preferably applied by means of a pump atomizer. A pump atomizer containing a deodorizing cosmetic agent according to an exemplary embodiment is therefore a further subject matter of the present application.

The deodorizing agent may also be prepared as a roll-on. Such a preparation is particularly preferred along with the preparation as a pump atomizer. Application may take place for example by means of a roller ball applicator. Such rollers include a ball mounted in a ball bed, which ball can be moved by movement over a surface. In doing so, the ball takes up some of the antiperspirant cosmetic agent according to an exemplary embodiment that is to be distributed, and conveys it to the surface to be treated. Roll-on applicators containing a deodorizing cosmetic agent according to embodiments of the present disclosure are particularly preferred.

A suitable alternative application form of the deodorizing cosmetic agents is the combination with a disposable substrate, for example a substrate selected from the group consisting of wipes, pads and wads. Particular preference is given to moist wipes, that is to say moist wipes which have been pre-prepared for the user and which are preferably packaged individually, as are well known for example from the field of glass cleaning or from the field of moist toilet tissues. Such moist wipes, which advantageously may also contain preservatives, have a deodorizing cosmetic agent according to an exemplary embodiment of the present disclosure impregnated therein or applied thereto and are preferably packaged individually. They may be used for example as a deodorant wipe, which is particularly beneficial for use on the go. Preferred substrate materials are selected from porous flat wipes. They may be made of a fibrous or cellular flexible material which has sufficient mechanical stability and at the same time softness for use on the skin. These wipes include wipes made of woven and nonwoven synthetic and natural fibers, felt, paper or foam, such as hydrophilic polyurethane foam. Deodorizing or antiperspirant substrates which are preferred according to an exemplary embodiment can be obtained by soaking or impregnation or also by melting an antiperspirant cosmetic agent according to embodiments of the present disclosure onto a substrate.

A further claimed subject matter is a non-therapeutic cosmetic method for preventing and/or reducing body odor, in which a cosmetic agent according to an exemplary embodiment is applied to the skin, in particular to the skin of the underarms, and remains on the skin for at least about 1 hour, preferably for at least about 2 hours, more preferably for at least about 4 hours, in particular for at least about 6 hours.

As mentioned in the introduction, the cosmetic agents according to an exemplary embodiment have an advantageous effect on the formation of body odor. The use of said cosmetic agent to prevent and/or reduce body odor is a further subject matter of the present application.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. An aqueous, deodorizing cosmetic agent comprising:
at least one quaternary ammonium compound, and
triethyl citrate,
wherein the deodorizing cosmetic agent contains no aluminum-containing compounds.

2. The deodorizing cosmetic agent according to claim 1, wherein the quaternary ammonium compound includes at least one compound selected from the group consisting of behentrimonium chloride, cetrimonium bromide, cetrimonium chloride, laurtrimonium bromide, laurtrimonium chloride, steartrimonium bromide, steartrimonium chloride, benzethonium chloride, benzalkonium chloride, and mixtures thereof.

3. The deodorizing cosmetic agent according to claim 1, wherein the agent contains benzalkonium chloride as the quaternary ammonium compound.

4. The deodorizing cosmetic agent according to claim 1, wherein the quaternary ammonium compound is present in an amount of from about 0.01 to about 0.3% by weight based on the total weight of the deodorizing cosmetic agent.

5. The deodorizing cosmetic agent according to claim 1, wherein the triethyl citrate is present in an amount of from about 0.1 to about 5.0% by weight based on the total weight of the deodorizing cosmetic agent.

6. The deodorizing cosmetic agent according to claim 1, wherein the agent includes at least one bactericide selected from the group consisting of chlorhexidine, farnesol, triclosan, chloroxylenol and phenoxyethanol.

7. The deodorizing cosmetic agent according to claim 1, wherein ethanol is present in an amount of from about 10 to about 50% by weight based on the total weight of the deodorizing cosmetic agent.

8. The deodorizing cosmetic agent according to claim 1, wherein water is present in an amount of from about 45 to about 85% by weight based on the total weight of the deodorizing cosmetic agent.

9. A method comprising using a deodorizing cosmetic agent according to claim 1 to prevent and/or reduce body odor.

10. A non-therapeutic cosmetic method for preventing and/or reducing body odor, the method comprising applying a cosmetic agent according to claim 1 to the skin, and allowing the cosmetic agent to remain on the skin for at least about 1 hour.

11. The deodorizing cosmetic agent according to claim 4, wherein the quaternary ammonium compound is present in an amount of from about 0.02 to about 0.2% by weight based on the total weight of the deodorizing cosmetic agent.

12. The deodorizing cosmetic agent according to claim 4, wherein the quaternary ammonium compound is present in an amount of from about 0.05 to about 0.1% by weight based on the total weight of the deodorizing cosmetic agent.

13. The deodorizing cosmetic agent according to claim 5, wherein the triethyl citrate is present in an amount of from about 0.25 to about 4% by weight based on the total weight of the deodorizing cosmetic agent.

14. The deodorizing cosmetic agent according to claim 5, wherein the triethyl citrate is present in an amount of from about 0.5 to about 3.0% by weight based on the total weight of the deodorizing cosmetic agent.

15. The deodorizing cosmetic agent according to claim 7, wherein ethanol is present in an amount of from about 20 to about 40% by weight based on the total weight of the deodorizing cosmetic agent.

16. The deodorizing cosmetic agent according to claim 7, wherein ethanol is present in an amount of from about 23 to about 35% by weight based on the total weight of the deodorizing cosmetic agent.

17. The deodorizing cosmetic agent according to claim 8, wherein water is present in an amount of from about 55 to about 75% by weight based on the total weight of the deodorizing cosmetic agent.

18. The deodorizing cosmetic agent according to claim 8, wherein water is present in an amount of from about 58 to about 70% by weight based on the total weight of the deodorizing cosmetic agent.
